(19) United States
(12) Patent Application Publication
(10) Pub. No.: US 2017/0326025 A1
(43) Pub. Date: Nov. 16, 2017

(54) VIBROTACTILE STIMULATION DEVICE

(71) Applicants: INSERM (INSTITUT NATIONAL DE LA SANTE ET DE LA RECHERCHE MEDICALE), Paris (FR); UNIVERSITE DE RENNES I, Rennes (FR)

(72) Inventor: Alfredo HERNANDEZ, Cesson-sevigne (FR)

(73) Assignees: INSERM (INSTITUT NATIONAL DE LA SANTE ET DE LA RECHERCHE MEDICALE), Paris (FR); UNIVERSITE DE RENNES I, Rennes (FR)

(21) Appl. No.: 15/529,202

(22) PCT Filed: Nov. 24, 2015
(86) PCT No.: PCT/IB2015/059084

§ 371 Date: May 24, 2017

(30) Foreign Application Priority Data
Nov. 24, 2014 (FR) 14/61375

(51) Int. Cl. A61H 23/02; A61B 5/0492; A61B 5/00; A61B 5/0408; A61B 5/087

(52) U.S. Cl. CPC A61H 23/0245; A61B 5/0408; A61B 5/0492; A61B 5/087; A61B 5/4818; A61B 5/4836; A61B 5/68335; A61H 2201/1604; A61H 2201/1619; A61H 2201/164; A61H 2201/165; A61H 2230/045; A61H 2230/405; A61H 2201/0188

(57) ABSTRACT

A vibrotactile stimulation device intended to be applied against a body medium (MC) to be stimulated, produced in the form of a functional unit, comprising a vibrating effector suitable for applying, to said medium, pulses of mechanical vibrational energy, and a controller for controlling the effector according to stimulation rules. The functional unit further houses a first electrode suitable for cooperating with at least one second electrode separated from the first electrode in order to supply signals representative of a cardiac activity and a muscular activity on the medium to be stimulated, said controller being sensitive to cardiac activity and muscular activity signals in order to influence the stimulation. The stimulation device may be used for body stimulation in combating sleep apnea, with improved detection.

### FIELD OF THE INVENTION

The present invention concerns vibrotactile or kinesthesic stimulation devices in general.

### PRIOR ART

Vibrotactile or kinesthesic stimulation is known in the prior art and has been used routinely for more than 20 years in various fields of application, especially for sensory assistance.

Thus, document U.S. Pat. No. 5,035,242 A concerns a stimulation device for the hearing impaired, and document WO 2009082682 A1 proposes a device for those with vision defects.

Vibrotactile stimulation is also used for treatment of sleep apnea, especially in premature newborns (see in particular U.S. Pat. No. 5,555,891 A or WO2007141345 A1).

Several transducer principles are used in vibrotactile or kinesthesic effectors. In the vast majority, these provide a mechanical vibrational stimulation by the application of an electric signal of predefined characteristics.

In general, the control of the mechanical vibrational stimulation is effectuated in response to the detection of apnea, typically by determining the respiratory air flow in the nose and/or the mouth of the subject thanks to a cannula connected to the device.

This basic principle may be sufficient in itself to abolish the apnea phase, but it is found today that the characteristics of the stimulation would benefit from taking other biological factors into account.

### SUMMARY OF THE INVENTION

The present invention intends to propose a vibrotactile stimulation device, especially but not exclusively for the treatment of sleep apnea, which is also able to estimate at least one other parameter which may be taken into account to adjust the stimulation.

Thus, according to the invention, a vibrotactile stimulation device is proposed, especially to effect a bodily stimulation in the fight against sleep apnea, designed to be applied against a body medium being stimulated, realized in the form of a functional unit comprising a vibrating effector able to apply to said medium pulses of mechanical vibrational energy, and means of control of the effector depending on the rules of stimulation, the device being characterized in that the functional unit also houses a first electrode able to cooperate with at least one second electrode at a distance from the first electrode to furnish signals representative of a cardiac activity and a muscular activity on the medium being stimulated, said control means being sensitive to the cardiac activity signals on the one hand and the muscular activity signals on the other hand to influence the stimulation.

These characteristics may be supplemented with the following preferred yet optional characteristics, taken in every combination which the person skilled in the art might find to be technically compatible.
the device comprises two units, namely, a first unit housing said effector and the first electrode, and a second unit housing the at least one second electrode.
the first unit or the second unit houses a third electrode forming a reference electrode.
the two units are joined together by a bundle of conductors.
the device further comprises a nasal cannula for the detection of a respiratory air flow, and the bundle of conductors is associated with the nasal cannula.
the device comprises a processing circuit able to derive from the signals furnished by the electrodes at least information on cardiac activity or information on muscular activity in the region of the electrodes, such as the mastoid region.
the processing circuit is able to make a separation between electrocardiogram signals and electromyogram signals.
the control means are able to control the effector as a function of the information on activity delivered by the processing circuit.
each unit comprises a plate carrying its various elements, and a disposable flexible casing able to receive the plate in removable manner.
the collection of elements carried by each plate are encapsulated.
each flexible casing comprises features able to receive and hold elastically at least part of the edge of the associated unit.
each casing has a peripheral edge provided with an adhesive.

### BRIEF DESCRIPTION OF THE DRAWINGS

Other aspects, purposes and advantages of the present invention will appear better upon perusal of the following detailed description of a preferred embodiment thereof, given as a nonlimiting example and making reference to the enclosed drawings, in which:

FIG. 1 is a schematic front view of a vibrotactile stimulation device made up of two units, designed to be placed respectively behind the ears of a patient, and the various elements making up this device,

FIG. 2 is a schematic front view of the head of a subject equipped with the two parts and an electrical connection element,

FIG. 3 shows an example of signals received with the device, and

FIGS. 4a and 4b are cross sectional views of the two units equipped with a means of fixation and protection.

### DETAILED DESCRIPTION OF A PREFERRED EMBODIMENT

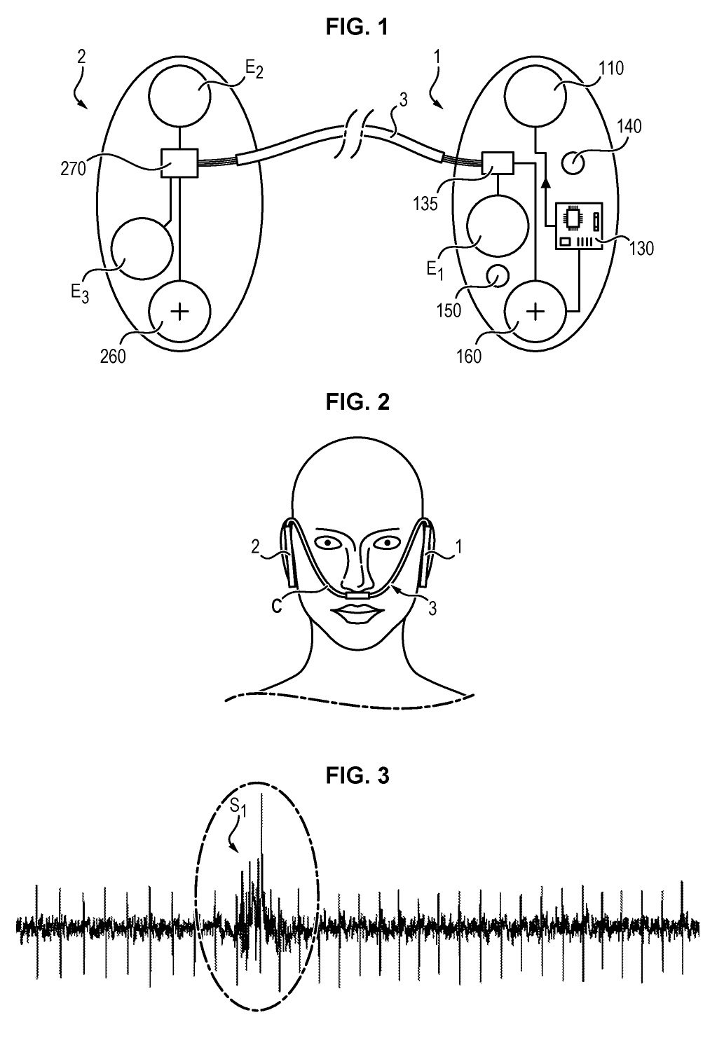

Making reference to FIGS. 1 and 2, a vibrotactile or kinesthesic stimulation device 1 according to the invention comprises two units 1 and 2, designed here to be placed behind the two ears of a subject.

A first unit 1 comprises an effector or electromechanical vibrational exciter 110. Preferably, the effector 110 comprises a piezoelectric element or a linear resonant actuator. The unit 1 likewise comprises on-board digital processing means 130, typically in the form of an electronic circuit card provided with a microcontroller, designed to control the effector as a function of certain parameters determining in particular (i) the presence of a sleep apnea phenomenon in the subject, (ii) the changes in heart rate and (iii) characteristics extracted from the electromyogram (electrical muscular activity) of the muscles in the neck.

Referring more particularly to FIG. 2, the device is able to emit mechanical pulses of excitation as a function of criteria such as the detecting of a sleep apnea phenomenon by means which may be of conventional type, and in particular the detecting of a respiratory air flow determined by a nasal cannula C, as illustrated schematically in FIG. 2.

These pulses for example have a frequency on the order of 100 to 400 Hz, and they are controlled by the control circuit 130 in response to the detection signals received via a wire-line or wireless interface, not shown.

The unit 1 may have other functionalities. For example, it may integrate a temperature sensor 140 providing temperature signals, either analog and converted into digital signals in the area of the processing device or directly digital, a light (LED) and/or sonic signaling device (vibrator) 150 indicating the state of the device, an on/off switch, etc.

It is energized by a button cell 160 having an appropriate capacity, or optionally by a rechargeable battery, in wired manner (for example, to the USB port of a computer) or wirelessly (by inductive power transmission, in known manner for small electronic appliances).

According to one variant embodiment, the digital processing means, or a portion of these processing means, may be moved to a separate box of the device and either be carried by the patient or arranged in proximity to the patient, for example, on their nightstand, during sleep.

Means of transmission are then provided to enable the communication of the device with the remote box, these means being either wire-line or wireless for a box carried by the patient, and preferably wireless for a fixed box.

According to one characteristic of the invention, the device is likewise able to detect cardio-respiratory phenomena which can be used by the control unit 130 to estimate the times at which a stimulation by means of the effector 110 is needed and to determine the parameters of this stimulation.

For this purpose, the device comprises a set of electrodes designed to be in contact with the skin of the patient when the units are put in place.

A first electrode E1 is situated in the unit 1, while two other electrodes E2 and E3 are situated in a second unit designated by reference number 2, designed to be placed on the subject at a distance from the first unit 1. For example, the second unit 2 may be applied to the mastoid bone behind the right ear of the patient, when the unit 1 is placed on the mastoid bone behind the left ear, or vice versa.

The electrodes E2 and E3 of the unit 2 are connected electrically to the unit 1 such that a processing circuit for signals furnished by the electrodes, designated by reference number 135, can analyze these signals and provide an indication of cardiac and/or respiratory activity of the subject. Advantageously, this electrical connection is realized with the aid of a set of conductors 3 associated with a nasal cannula C used, in known manner, to estimate an air flow in the vicinity of the nostrils and the mouth of the subject and to deduce from this the existence of a respiratory activity of the subject, or lack thereof.

These conductors may be integrated in a wall of the cannula C, or be secured to it in any other way.

As a variant, the conductors 3 may be arranged behind the head of the subject, for example, integrated in an elastic band or held by such a band.

When the units are placed behind the two ears, as illustrated in FIG. 2, these electrodes are able to collect a blend of electrophysiological signals principally reflecting the electrical cardiac activity and activity of the muscles near the mastoid region.

One of the electrodes, such as electrode E3 situated in the unit 2, is a reference electrode, while the signals collected by the electrodes E1 and E2 at a distance from each other are applied to the inputs of a differential high-gain amplifier provided in the processing circuit 135.

FIG. 3 shows an example of the appearance of the signals collected, reflecting the aforementioned blend of activities. Peaks distributed in rather regular fashion are observed, corresponding to the cardiac pulses (R wave of the electrocardiogram ECG). An electrical activity of the surrounding muscles (EMG) shown by a signal of lesser amplitude and more elevated frequency content is also observed, occurring for the entire extent of the sample shown. This EMG signal is liable to hide the P and T waves of the ECG. In particular, the perturbation (signal zone S1) illustrated in FIG. 3 corresponds to a significant increase in muscle activity in the mastoid region, as shown by the increase in energy of the EMG signal.

The processing circuit 135 is able to mutually separate these ECG and EMG signals in order to process them distinctly. For example, it carries out a method of separation of sources or adaptive linear or nonlinear filtering, which is known in itself.

Once the signals have been separated, the resulting EMG signal can be used to estimate the level of tone of the surrounding muscles. In particular, it has been found that a decrease in tone of the muscles of the pharynx plays an important role in the genesis of obstructive apneas. On the other hand, a resumption of muscle tone (such as that shown by the signal portion S1 in FIG. 3) is often associated with the end of an obstructive apnea. The muscle tone thus estimated likewise provides information as to the sleep condition of the patient, particularly so as to differentiate between sleep and wake states.

The processing of signals according to this aspect of the invention thus enables an analyzing of this muscle tone in order to (i) activate the stimulation therapy at an early stage, (ii) differentiate between central apneas and obstructive apneas, (iii) control the stimulation in optimal manner, and (iv) optimize the parameters for detecting of respiratory events, for example as a function of the sleep states.

As for the ECG signal resulting from the separation, the principal information extracted is the heart rate, it being known to be highly perturbed during episodes of apnea, and this is used in known manner as a control variable enabling the optimization of the vibrotactile stimulation (see WO2007141345 A1 already cited).

The circuit 135 is functionally connected to the control circuit 130 of the vibrotactile stimulation, so as to be able to adjust the stimulation strategies as a function not only of the respiratory activity proper, as measured by the cannula C and optionally by other sensors, but also of the cardiac rhythm of the subject and the muscle tone in the mastoid region, as indicated above. It will be noted that the processing for separation of the signals coming from the electrodes (particularly filtering, frequency breakdown, detection of amplitude, etc.) may be done either in the circuit 135, or in the circuit 130. Furthermore, the person skilled in the art will understand that the circuits 130 and 135 may be merged into a single circuit.

The conductors 3 preferably comprise at least two conductors, namely, one for each electrode E2 and E3.

The different functions of the device may furthermore be apportioned between the two units 1 and 2, and in particular the unit 2 may likewise comprise a processing or control circuit, a battery, signaling devices, an on/off switch, etc.

Of course, the number of conductors in the bundle 3 will be adapted by the skilled person as a function of the apportionment of functions between the two units.

Furthermore, if the space in the unit 1 is sufficient, the reference electrode E3 could be integrated there.

The dimensions of each unit of the device are typically 3 to 8 cm along the major axis and 2 to 6 cm along the minor axis.

It should also be noted that the unit 2 may be put in any place other than behind the ear opposite the one where the unit 1 is located, it being noted that in order to gather the appropriate signals on cardiac activity and/or muscle activity the distance between the electrodes E1 and E2 should be large enough (typically at least 10 to 20 cm).

The nature of the connection between the two units 1 and 2 will then be adapted accordingly.

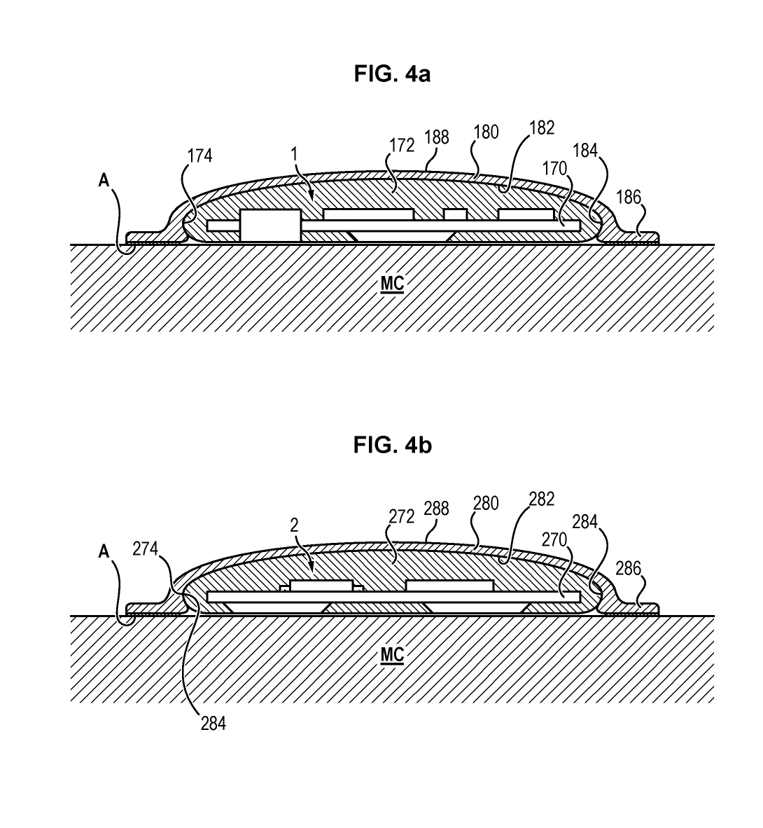

FIGS. 4a and 4b illustrate a practical implementation of the device according to the invention. The various elements of each unit 1, 2 are mounted on a plate, respectively 170, 270, the whole being encased in a block or shell, respectively 172, 272, for example one made of resin, having peripheral edges 174, 274 respectively suitable to the coupling with a means of fixation on the skin. Such a means of fixation may be a disposable casing 180, 280 respectively, made of an elastic material and able to receive the respective unit 1, 2 in a respective internal cavity 182, 282, holding it by the respective edges 174, 274 of the encapsulating resin block, which are engaged in a respective peripheral notch 184, 284 of said casing.

A biocompatible adhesive A may be provided at the respective peripheral edge 186, 286 of each casing, designed to be in contact with the skin, while a top wall 188, 288 respectively of the casing covers and entirely seals the associated unit encapsulated in its respective resin block 172, 272.

It will be understood that the encapsulated unit 1 or 2 may be easily extracted from its casing 180, 280 in order to replace it with a new casing. The adhesive A may be covered, in conventional manner, by a protective film which can be peeled off prior to use.

The units forming the stimulation device according to the invention can be secured to any adapted site (behind the ears, on the lateral chest, the sole of the feet, etc.), the casings 180, 280 and their characteristics being then adapted to the intended use. Likewise, the electrical connection between the two units will be adapted as a consequence.

FIGS. 4a and 4b show that the electrodes are directly in contact with the medium being stimulated MC, whereas the effector 10 may apply the vibrations to the medium either by direct contact, or via the material of the shell 172, which is then chosen to ensure an appropriate mechanical coupling with the medium MC.

It may also be provided, in familiar fashion, that the surface of the electrodes is covered by a medium promoting the electrical conduction.

Of course, the invention is in no way limited to the embodiment described and represented, but rather the person skilled in the art will be able to add many variants and modifications to it.

In particular, any independent functionality or one correlated with the vibrational stimulation may be added to the device, and especially any detection or sensing of another biological parameter, besides the temperature measurement.

1. A vibrotactile stimulation device, especially to effect a bodily stimulation in the fight against sleep apnea, designed to be applied against a body medium being stimulated, realized in the form of a functional unit comprising:
a vibrating effector able to apply to said medium pulses of mechanical vibrational energy,
a controller for controlling the effector depending on the rules of stimulation, and
a first electrode able to cooperate with at least one second electrode at a distance from the first electrode to furnish signals representative of a cardiac activity and a muscular activity on the medium being stimulated, said controller being sensitive to the cardiac activity signals on the one hand and the muscular activity signals on the other hand to influence the stimulation.

2. The device as claimed in claim 1, further comprising a first unit housing said vibrating effector and the first electrode, and a second unit housing the at least one second electrode.

3. The device as claimed in claim 2, wherein the first unit or the second unit houses a third electrode forming a reference electrode.

4. The device as claimed in claim 2, wherein the two units are joined together by a bundle of conductors.

5. The device as claimed in claim 4, further comprising a nasal cannula for the detection of a respiratory air flow, and wherein the bundle of conductors is associated with the nasal cannula.

6. The device as claimed in claim 1, wherein it comprises a processing circuit able to derive from the signals furnished by the electrodes at least information on cardiac activity or information on muscular activity in the region of the electrodes, such as the mastoid region.

7. The device as claimed in claim 6, wherein the processing circuit is able to make a separation between electrocardiogram signals and electromyogram signals.

8. The device as claimed in claim 6, wherein the control means are able to control the effector as a function of the information on activity delivered by the processing circuit.

9. The device as claimed in claim 1, wherein each unit comprises a plate carrying its various elements, and a disposable flexible casing able to receive the plate in removable manner.

10. The device as claimed in claim 9, wherein the collection of elements carried by each plate are encapsulated.

11. The device as claimed in claim 9, wherein each flexible casing comprises features able to receive and hold elastically at least part of the edge of the associated unit.

12. The device as claimed in claim 9, wherein each casing has a peripheral edge provided with an adhesive.
